(19) United States
(12) Patent Application Publication
(10) Pub. No.: US 2012/0177601 A1
(43) Pub. Date: Jul. 12, 2012

(54) TREATMENT OF HEPATITIS C VIRUS INFECTIONS

(75) Inventor: Warren Schmidt, Oxford, IA (US)

(73) Assignees: THE USA AS REPRESENTED BY THE SECRETARY OF THE DEPARTMENT OF VETERANS AFFAIRS, Washington, DC (US); THE UNIVERSITY OF IOWA RESEARCH FOUNDATION, Iowa City, IA (US)

(21) Appl. No.: 13/381,548

(22) PCT Filed: Jun. 9, 2010
(86) PCT No.: PCT/US10/37970

§ 371 Date: Mar. 21, 2012

Related U.S. Application Data
(60) Provisional application No. 61/222,761, filed on Jul. 2, 2009.

(51) Int. Cl. A61K 31/4025; A61P 31/14; A61K 31/708; C12N 5/071; A61K 38/21

(52) U.S. Cl. 424/85.4; 514/422; 435/375; 514/45

(57) ABSTRACT

This present invention provides for a new class of HCV NS3/4A protease inhibitors as additional therapeutics for hepatitis C virus. The proposed compounds, biliverdin, bilirubin, and derivatives thereof, are based on natural enzymatic products of heme metabolism that may be more stable, better tolerated, and more resistant to mutations than present prototypic protease inhibitors.

The present application claims benefit of priority to U.S. Provisional Application Ser. No. 61/222,761, filed Jul. 2, 2009, the entire contents of which are hereby incorporated by reference.

This invention was made with government support under grant number NIH DK068453 awarded by the National Institutes of Health. The government has certain rights in the invention.

### BACKGROUND OF THE INVENTION

A. Field of the Invention

The invention is directed to the fields of infectious disease, virology and medicine. More specifically, the invention is directed at compositions and methods for the treatment of Hepatitis C Virus (HCV) infections.

B. Description of the Related Art

HCV is a (+) sense RNA virus of the Hepacivirus genus and the flaviviridae family. Chronic hepatitis C virus (HCV) infection causes liver disease, cirrhosis, and hepatocellular carcinoma in over 175 million persons worldwide. Also, the currently approved treatment for HCV is a rigorous course of 24 to 48 weeks of pegylated interferon in combination with ribavirin. Moreover, combination therapy has numerous side effects and is only effective in about 50% of treated individuals.

In HCV, there are at least 3 structural and 6 non-structural proteins which are initially encoded as a large polyprotein translated early in the viral life cycle. Individual non-structural proteins are then formed after the polyprotein is cleaved at selective points by a specific viral protease, designated as NS 3/4A. Not surprisingly, because functional HCV protease activity is crucial to the success of the viral life cycle, considerable effort has been expended to prepare HCV protease inhibitors for use as antiviral drugs.

HCV NS3/4A protease is a member of the chymotrypsin family of serine-activated proteases. As such, the enzymatic active site of chymotrypsin was initially used to model potential antiprotease drugs for HCV. Recently, relatively successful examples of inhibitors (e.g., boceprevir, telaprevir) have proceeded to phase II clinical trials and appear promising for candidates with eventual FDA approval. However, the chance for development of resistance to these drugs and other current antiviral proteases is high, and the search for new and successful antiproteases must proceed onward. Moreover, there remains no HCV vaccine available, due to genetic variability and impaired adaptive immunity being the two major obstacles. Consequently, research interest continues to focus new treatment modalities to overcome these shortcomings.

### SUMMARY OF THE INVENTION

Thus, in accordance with the present invention, there is provided a method for inhibiting hepatitis C virus (HCV) replication comprising contacting an HCV-infected cell with bilirubin, biliverdin and/or a biliverdin derivative. The biliverdin derivative may have the structure:

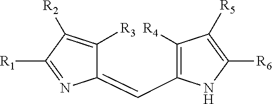

wherein:
R₁ and R₆ are independently alkenyl_{(C≦12)}, aryl_{(C≦12)}, aralkyl_{(C≦12)}, heteroaryl_{(C≦12)}, heteroaralkyl_{(C≦12)}, acyl_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups; and
R₂, R₃, R₄ and R₅ are independently:
   hydrogen, hydroxy, halo, amino, nitro, hydroxyamino, cyano, azido or mercapto; or
   alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, alkenyloxy_{(C≦12)}, alkynloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups;
      or a pharmaceutically acceptable salt, tautomer, or optical isomers thereof, provided that the biliverdin derivative is not biliverdin. The method may further comprise contacting said cell with second agent selected from the group consisting of pegylated interferon, ribavarin or an NS3/4A protease inhibitor. The second agent may be contacted with said cell at the same time as, before or after bilirubin, biliverdin or a biliverdin derivative. The cell may be contacted with bilirubin, biliverdin or a biliverdin derivative at least a second time. In particular, the cell may be contacted with (i) bilirubin and biliverdin; (ii) bilirubin and a biliverdin derivative; (iii) biliverdin and a biliverdin derivative; or (iv) bilirubin, biliverdin and a biliverdin derivative.

In another embodiment, the present invention provides a method for inhibiting hepatitis C virus (HCV) replication in a subject comprising administering to said subject bilirubin, biliverdin or a biliverdin derivative. The biliverdin derivative may have the structure:

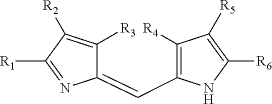

wherein:
R₁ and R₆ are independently alkenyl_{(C≦12)}, aryl_{(C≦12)}, aralkyl_{(C≦12)}, heteroaryl_{(C≦12)}, heteroaralkyl_{(C≦12)}, acyl_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups; and
R₂, R₃, R₄ and R₅ are independently:
   hydrogen, hydroxy, halo, amino, nitro, hydroxyamino, cyano, azido or mercapto; or
   alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, alkenyloxy_{(C≦12)}, alkynyloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups;
      or a pharmaceutically acceptable salt, tautomer, or optical isomers thereof, provided that the biliverdin derivative is not biliverdin. The method may further comprise administering to said subject a second agent selected from the group consisting of pegylated interferon, ribavarin or an NS3/4A protease inhibitor. The second agent may be administered at the same time as, before or after bilirubin, biliverdin or a biliverdin derivative. The method may further comprise administering to said subject bilirubin, biliverdin or a biliverdin derivative at least a second time. In particular, the cell may be contacted with (i) bilirubin and biliverdin; (ii) bilirubin and a biliverdin derivative; (iii) biliverdin and a biliverdin derivative; or (iv) bilirubin, biliverdin and a biliverdin derivative.

In yet another embodiment, there is provided a pharmaceutical formulation comprising (a) bilirubin, biliverdin and/or a biliverdin derivative; and (b) pegylated interferon, ribavarin and/or an, NS3/4A protease inhibitor, dispersed in a pharmaceutically acceptable buffer, diluent or excipient. The biliverdin derivative may have the structure:

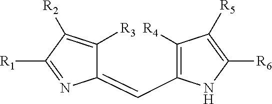

wherein:
R₁ and R₆ are independently alkenyl_{(C≦12)}, aryl_{(C≦12)}, aralkyl_{(C≦12)}, heteroaryl_{(C≦12)}, heteroaralkyl_{(C≦12)}, acyl_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups; and
R₂, R₃, R₄ and R₅ are independently:
   hydrogen, hydroxy, halo, amino, nitro, hydroxyamino, cyano, azido or mercapto; or
   alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, alkenyloxy_{(C≦12)}, alkynyloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups;
      or a pharmaceutically acceptable salt, tautomer, or optical isomers thereof, provided that the biliverdin derivative is not biliverdin. The formulation may comprise (i) biliverdin, pegylated interferon and ribavarin, (ii) bilirubin, pegylated interferon and ribavarin, or (iii) a biliverdin derivative, pegylated interferon and ribavarin.

As used herein, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylamino); “cyano” means —CN; “azido” means —N₃; “phosphate” means —OP(O)(OH)₂; “mercapto” means —SH; “thio” means ═S; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); “sulfinyl” means —S(O)— (see below for definitions of groups containing the term sulfinyl, e.g., alkylsulfinyl); and “silyl” means —SiH₃ (see below for definitions of group(s) containing the term silyl, e.g., alkylsilyl).

For the groups below, the following parenthetical subscripts further define the groups as follows: “(Cn)” defines the exact number (n) of carbon atoms in the group. “(C≦n)” defines the maximum number (n) of carbon atoms that can be in the group, with the minimum number of carbon atoms in such at least one, but otherwise as small as possible for the group in question. E.g., it is understood that the minimum number of carbon atoms in the group “alkenyl_{(C≦8)}” is 2. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3-10 carbon atoms)). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3-10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

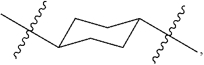

are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “aryl” when used without the “substituted” modifier refers to a monovalent group with an aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group with an aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S, Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur.

Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not

carbon or hydrogen, beyond the oxygen atom of the carbonyl group. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above. For example, —OCH₂CF₃ is a substituted alkoxy group.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group ═NR, wherein the alkylimino group is attached with one σ-bond and one π-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one σ-bond and one π-bond, in which R is substituted alkenyl, alkynyl, aryl, aralkyl, heteroaryl, heteroaralkyl and acyl, respectively.

Any undefined valency on an atom of a structure shown in this application implicitly represents a hydrogen atom bonded to the atom.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use (2002).

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

“Therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein the specification, “a” or “an” may mean one or more. As used herein in the claim(s), when used in conjunction with the word “comprising,” the words “a” or “an” may mean one or more than one. As used herein “another” may mean at least a second or more. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Biliverdin and bilirubin suppression of HCV replication. Log-phase full-length (FL) (FIG. 1A) or nonstructural (NS) (FIG. 1B) replicon cells were treated 48 hr with indicated concentrations of BV or BR. HCV replication was determined by real-time RT-PCR using the comparative cycle threshold (ΔC_{T}) method. ** or ## or ̂̂ p<0.01 from respective controls. ̂ p<0.05 from control.

FIGS. 2A-D. Effect of BV, BR, and FeCl₂ treatment on HCV proteins. (FIGS. 2A-B) Log-phase NS or FL replicons were treated with biliverdin (20 μM), BR (200 μM) or FeCl₂ (100 μM) for 24 hr. Cells were then lysed and protein expression evaluated by WB. (FIGS. 2C-D) Full-length replicons were treated overnight with various concentrations of BV and assayed by WB (FIG. 2C) or immunoprecipitation for NS5A (FIG. 2D). In FIG. 2D, upper band was identified as NS5A and lower band was immunoglobulin heavy chain (Ig HC).

FIGS. 3A-D. Biliverdin inhibition of J6/JFH HCV replication in Huh7.5 cells. (FIGS. 3A-C) J6/JFH infected Huh7.5 cells were treated with different concentrations of BV (0, 20, 200 μM, FIGS. 3A-C respectively) for 72 hr. Cultures were then fixed and stained immunocytochemically with HCV genotype 2A polyvalent human serum as described in methods. (FIG. 3D) HCV RNA was quantified using comparative threshold (ΔC_{T}) assay. **HCV RNA in BV-treated cells versus control (p<0.01).

FIGS. 4A-C. Inhibition of NS3/4A protease. (FIGS. 4A-B) Protease activity was determined fluorometrically using recombinant NS3/4A enzyme as described in Methods. (FIG. 4C) Endogenous NS3/4A protease activity in microsomes of replicons was measured using the same assay. Inhibitor refers to the commercial NS3/4A protease competitive inhibitor, AnaSpec #25346. BV=>99% Biliverdin IX-α. BR-mixed isomers (MI)=93% Bilirubin IX-α, and 6% associated BR isomers as described in Materials. BR-IXα=>99% bilirubin IX-α.

FIGS. 5A-C. Kinetics of BV inhibition of NS3/4A protease. (FIG. 5A) Reciprocal (Lineweaver-Burk) plot of substrate concentration versus enzyme activity. Recombinant protease activity was determined fluorometrically as described in FIG. 4 and Methods. (FIGS. 5B-C) Secondary plots of 1/Vap (y-intercepts) or Km/V (slopes) versus BV concentrations to estimate Ki′ and Ki of BV inhibition respectively. Plot of [BV] vs either 1/Vap or Km/V showed highly significant linearity, (r=0.975 and r=0.979 respectively, P<0.005) suggesting mixed inhibition of NS3/4A protease by BV (Ki′=1.1 and Ki=0.6 μM, respectively).

FIGS. 6A-B. Effect of biliverdin reductase (BVR) knockdown on antiviral activity. (FIG. 6A) The efficiency of BVR knockdown was determined by WB after transfection of BVR siRNA or scrambled controlled RNA into NS (left panel) or FL (right panel) replicons. Real-time RT-PCR measurements for HCV RNA were performed after control vehicle or BV (20 μM) overnight incubation in both replicon lines (FIG. 6B, left panel). Note that the antiviral activity of BV was significantly enhanced (P<0.01) when BVR was knocked down. In FIG. 6B (right panel) HCV RNA was quantified after control vehicle or BR (100 μM) overnight incubation in both replicon lines. BVR knockdown had no effect on the antiviral activity of BR. ## not significant; *p<0.01; **p<0.005.

FIGS. 7A-B. Additive effect of BV on α-interferon antiviral activity. FL (FIG. 7A) or NS (FIG. 7B) replicons were treated with indicated amounts of α-interferon alone or in the presence of 50 or 100 μM BV overnight. HCV replication was determined by real-time RT-PCR using the comparative cycle threshold (ΔC_{T}) method. (*) p<0.01 vs. control; (#) p<0.005 vs. control.

### DETAILED DESCRIPTION OF THE INVENTION

Current treatment for chronic HCV is only successful long term in about 50% of all treated individuals. In pilot clinical studies, the addition of an anti-protease drug to the established treatment regimen resulted in increased incidence of long term remission from the virus. However, current prototypic HCV protease inhibitors are prone to resistance mutations by the virus (Kuntzen et al., 2008), and occur consistently in 1-8% of patients with “hard to treat” genotype 1 infections.

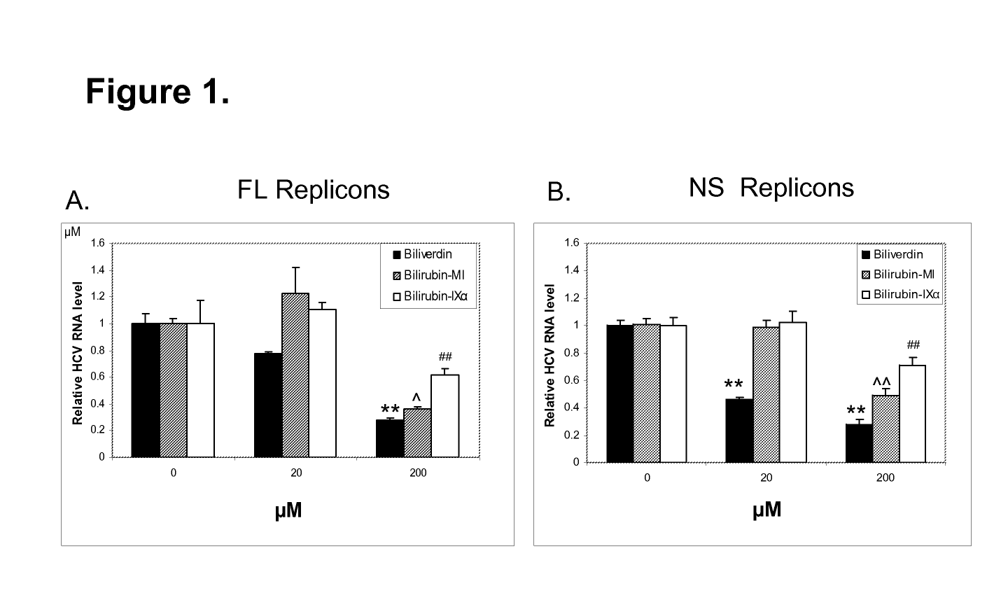

Heme oxygenase (HO) is a vital enzyme, responsible for the catalysis of heme and liberation of equimolar ratios of Fe⁺², carbon monoxide (CO), and biliverdin (Immenschuh and Ramadori, 2000; Ryter and Tyrrell, 2000). In the next reaction, biliverdin (BV) is rapidly converted to bilirubin (BR) by biliverdin reductase (FIG. 1) (Ryter et al., 2006). The reaction uses 3 moles of oxygen and reducing equivalents from NADPH:cytochrome P-450 (cytochrome c) reductase to proceed.

The inventors have reported that induction of heme oxygenase-1 (HO-1), an enzyme that oxidizes the porphyrin heme from senescent red blood cells, with heme or overexpression of the enzyme in HCV replicon cells inhibited viral replication. Heme oxidation by HO liberates equimolar amounts of BV, carbon monoxide, and Fe⁺² which are recycled inside liver cells. Of the HO-1 reaction products, free Fe⁺² was reported to inhibit the HCV RNA dependent RNA polymerase (RdRp). The inventors suspected that part of this antiviral activity was due to released Fe⁺² from the HO reaction (Zhu et al., 2008). Fillebeen et al. (2007) have demonstrated that iron can inhibit the HCV NS5B RdRp by competitive binding to the divalent cation binding pocket of the polymerase. Either Mg⁺⁺ or Mn⁺⁺ are absolutely required for NS5B enzymatic activity (Ferrari et al., 1999). None of the other HO reaction products, including BV and BR, were known to have antiviral activity.

The inventors have now discovered that BV and to a lesser extent, BR are potent inhibitors of the NS3/4A protease and that this activity occurs at physiological or low pharmacological concentrations of these agents in vitro. These findings indicate that either compound, or their active chemical derivatives, could be used as direct or adjuvant therapy for chronic HCV infection in conjunction with other established antiviral agents such as pegylated interferon and ribavirin. Moreover, BV and BR are natural heme breakdown products with a novel activity inhibitory for the HCV NS3/4A viral protease. Using natural compounds reduces the viruses' mutagenic capabilities during therapy. Further, because these compounds are normally metabolized and used by the liver, they are less like to exhibit adverse side effects. Additionally, it appears that these agents can be employed at near physiological or low pharmacological concentrations.

These and other aspects of the invention are described in detail below.

### I. HCV

A. Background

HCV is an enveloped single-stranded, positive-sense RNA virus classified into the Flaviviridae family (Choo et al., 1989). HCV has been well-documented as the major etiological agent responsible for most post-transfusional and community-acquired hepatitis (Alter et al., 1999). HCV results in persistent infection in up to 80% of infected individuals and causes a wide spectrum of liver diseases, including cirrhosis and hepatocellular carcinoma (Di Bisceglie, 1998). HCV-related liver disease is now the leading cause of liver transplantation in the United States. The Centers for Disease Control and Prevention have estimated that HCV causes 8,000˜10,000 deaths each year, with deaths expected to more than triple over the next two decades, eventually exceeding those from acquired immunodeficiency syndromes (Natl. Institutes of Health Consensus Dev. Conf. Panel, 1997).

The HCV virion contains a lipid envelope studded with viral envelope proteins that surrounds a protein capsid. Within the capsid is the viral genome comprised of a ˜9600 bp RNA molecule which is divided into three regions. The 5′-untranslated region (UTR) of the genome is highly structured and contains an internal ribosome entry site that permits efficient translation from the uncapped RNA genome. The 5′-UTR is followed by a single large open reading frame that encodes a single polyprotein of approximately 3010 amino acids. This polyprotein is processed into at least 10 functional proteins by host and viral proteases (Blight and Rice, 1997). Finally, there is a 200˜300 bp 3′-UTR composed of a variable sequence of about 40 bp, a polyU sequence of variable length, a polypyrimidine tract, and a high conserved 98 bp terminal sequence that folds into a highly conserved structure (Kolykhalov et al., 1996; Yamada et al., 1996).

The HCV genome is highly variable. Based on the phylogenetic analysis of nucleotide sequences, HCV is divided into at least six major genotypes (20-30% sequence difference) and more than 50 subtypes (10-20% sequence difference) (Robertson et al., 1998). Moreover, even within an individual infected with a single HCV subtype, HCV circulates as a group of different but genetically closely related variants, referred to as viral quasi-species (less than 10% sequence difference), a characteristic shared by most of RNA viruses (Eigen, 1993). The molecular basis for HCV quasi-species nature is the high viral turn-over rate (about 10¹² virions per day) (Neumann et al., 1998) and the high error rate of its RNA-dependent RNA polymerase encoded by HCV NS5B, which lacks proof-reading repair activity (Holland et al., 1982). Although the variability has been well documented across the entire HCV genome (Simmonds, 1995), the most variable regions are located on envelope domains. In particular, the 5′ end of the second envelope sequence, an 81 bp domain, has been proved to be extremely variable, named hypervariable region 1 (HVR1) (Hijikata et al., 1991; Kato et al., 1992).

Genetic variability has multiple implications for HCV pathogenesis and vaccine development. First, it allows the production of escape mutants in face of human immune system or antiviral therapies. HCV mutants with nucleotide substitutions either in B cell or in cytotoxic T lymphocyte (CTL) epitopes have been observed during chronic HCV infection (reviewed in Moorman et al., 2001; Rosenberg, 1999). Second, it facilitates the adaptation to new replication sites. For instance, the inventor found that HCV may adapt its replicative capability to a new host (the donor liver) by rapidly mutating the HVR1 domain in the setting of liver transplantation (Fan and Di Bisceglie, 2003). Third, it induces “original antigen sin” (OAS), a well-known immune phenomenon first described in influenza virus infection in 1950's (Fenner et al., 1974). OAS predicts weakened antibody responses in both concentration and affinity against HCV mutants, which facilitates the establishment of persistent HCV infection (Shimizu et al., 1994). OAS has also been observed in cellular immunity (Klenerman and Zinkernagel, 1998) and represents a major challenge in vaccine development for viruses with great genetic heterogeneity. Finally, because the dynamics of the immune response differ greatly for different HCV genotypes/subtypes (Yoshioka et al., 1997), it is difficult to select a vaccine strain.

There is accumulating evidence indicating that HCV infection does induce neutralizing antibodies, which play a partial role in the protection of HCV re-infection: (a) in patients with acute or chronic HCV infection, the natural resolution of HCV infection strongly correlates with the titers of putative neutralizing antibodies, anti-HVR1 (Ishii et al., 1998; Zibert et al., 1997); (b) chimpanzees inoculated with recombinant HCV E1/E2 heterodimer can be protected from experimental challenge with homologous virus (Choo et al., 1994). Putative neutralizing antibodies were detected in vaccinated chimpanzees but not in the control group (Lagging et al., 1998); (c) HCV-specific polyclonal globulins, purified from pools of human plasma that have high level of antibodies to HCV but normal ALT activities, may be capable of modifying the course of HCV infection and suppressing HCV replication in chimpanzees (Lemon et al., 2000), and post-exposure HCV immune globulin (HCIG) treatment also markedly prolonged the incubation period of acute hepatitis C (Krawczynski et al., 1996); and (d) in an epidemiological study with injecting drug users who are the high risk for HCV infection, the incidence of HCV infection was significantly lower in individuals with previous HCV infection than in people without previous HCV infection (Mehta et al., 2002).

C. Treatments

There is a very small chance of clearing the virus spontaneously in chronic HCV carriers (0.5 to 0.74% per year); however, the majority of patients with chronic hepatitis C will not clear it without treatment. Current treatment is a combination of pegylated interferon a (brand names PEGASYS™ and PEG-Intron) and the antiviral drug ribavirin for a period of 24 or 48 weeks, depending on genotype. Indications for treatment include patients with proven hepatitis C virus infection and persistent abnormal liver function tests.

Sustained cure rates (sustained viral response) of 75% or better occur in people with genotypes HCV 2 and 3 in 24 weeks of treatment, about 50% in those with genotype 1 with 48 weeks of treatment and 65% for those with genotype 4 in 48 weeks of treatment. About 80% of hepatitis C patients in the United States have genotype 1. Genotype 4 is more common in the Middle East and Africa. Should treatment with pegylated ribivirin-interferon not return a 2-log viral reduction or complete clearance of RNA (termed “early virological response”) after 12 weeks for genotype 1, the chance of treatment success is less than 1%. Early virological response is typically not tested for in non-genotype 1 patients, as the chances of attaining it are greater than 90%. The mechanism of action is not entirely clear, because even patients who appear to have had a sustained virological response still have actively replicating virus in their liver and peripheral blood mononuclear cells. The evidence for treatment in genotype 6 disease is currently sparse, and the evidence that exists is for 48 weeks of treatment at the same doses as are used for genotype 1 disease. Physicians considering shorter durations of treatment (e.g., 24 weeks) should do so within the context of a clinical trial.

Treatment during the acute infection phase has much higher success rates (greater than 90%) with a shorter duration of treatment; however, this must be balanced against the 15-40% chance of spontaneous clearance without treatment. Those with low initial viral loads respond much better to treatment than those with higher viral loads (greater than 400,000 IU/mL). Current combination therapy is usually supervised by physicians in the fields of gastroenterology, hepatology or infectious disease.

The treatment may be physically demanding, particularly for those with a prior history of drug or alcohol abuse. It can qualify for temporary disability in some cases. A substantial proportion of patients will experience a panoply of side effects ranging from a ‘flu-like’ syndrome (the most common, experienced for a few days after the weekly injection of interferon) to severe adverse events including anemia, cardiovascular events and psychiatric problems such as suicide or suicidal ideation. The latter are exacerbated by the general physiological stress experienced by the patient.

Current guidelines strongly recommend that hepatitis C patients be vaccinated for hepatitis A and B if they have not yet been exposed to these viruses, as infection with a second virus could worsen their liver disease.

### II. BILIVERDIN AND BILIRUBIN

A. Biliverdin

Biliverdin is a green tetrapyrrolic bile pigment, and is a product of heme catabolism. It is the pigment responsible for the yellowish color in bruises. Biliverdin results from the breakdown of the heme moiety of hemoglobin in erythrocytes. Macrophages break down senescent erythrocytes and break the heme down into biliverdin, which normally rapidly reduces to free bilirubin. This breakdown occurs in bruises, which leads to a yellowish color. Biliverdin has been found in excess in the blood of humans suffering from hepatic diseases. Jaundice is caused by the accumulation of biliverdin or bilirubin (or both) in the circulatory system and tissues. Jaundiced skin and whites of the eyes are characteristic of liver failure.

While typically regarded as a mere waste product of heme breakdown, evidence has been mounting that suggests biliverdin—and other bile pigments—has a physiological role in humans.

Bile pigments such as biliverdin naturally possess significant anti-mutagenic and antioxidant properties and therefore fulfill a useful physiological function. Biliverdin and bilirubin have been shown to be potent scavengers of peroxyl radicals. They have also been shown to inhibit the effects of polycyclic aromatic hydrocarbons, heterocyclic amines, and oxidants—all of which are mutagens. Studies have even found that people with higher concentrations levels of bilirubin and biliverdin in their bodies have a lower frequency of cancer and cardiovascular disease.

A 1996 study by McPhee et al. suggested that biliverdin—as well as many other tetrapyrrolic pigments—may function as an HIV-1 protease inhibitor. Of the fifteen compounds tested, biliverdin and bilirubin were the most active and for the HIV protease showed nearly equivalent activities with Ki of 1 μM and 0.8 μm respectively. In vitro experiments showed that biliverdin and bilirubin competitively inhibited HIV-1 proteases at low micromolar concentrations and also reduced viral infectivity. However, when tested in cell culture with micromolar concentrations, it was found that biliverdin and bilirubin also reduced infectivity by blocking viral entry into cells. Results were found to be similar for HIV-2 and SIV. Further research is needed to confirm these results, and to examine if unconjugated hyperbilirubinemia has any effect on the progression of HIV infection.

Current research has suggested that the anti-oxidant properties of biliverdin and other bile pigments may also have a beneficial effect on asthma. This is because oxidative stress may play a vital role in the pathogenesis of asthma. A 2003 study found that asthma patients suffering from jaundice brought on by acute hepatitis B exhibited temporary relief of asthma symptoms. However, there could also have been confounding factors such as elevated levels of cortisol and epinephrine, so more research into this possibility is required.

B. Bilirubin

Bilirubin (formerly referred to as hematoidin) is the yellow breakdown product of normal heme catabolism. Heme is found in hemoglobin, a principal component of red blood cells. Bilirubin is excreted in bile, and its levels are elevated in certain diseases. It is responsible for the yellow color of bruises and the yellow discoloration in jaundice. It has also been found in plants.

Bilirubin consists of an open chain of four pyrrole-like rings (tetrapyrrole). In heme, by contrast, these four rings are connected into a larger ring, called a porphyrin ring. Bilirubin is very similar to the pigment phycobilin used by certain algae to capture light energy, and to the pigment phytochrome used by plants to sense light. All of these contain an open chain of four pyrrolic rings. Like these other pigments, bilirubin changes its conformation when exposed to light. This is used in the phototherapy of jaundiced newborns: the isomer of bilirubin formed upon light exposure is more soluble than the unilluminated isomer.

Bilirubin is created by the activity of biliverdin reductase on biliverdin. Bilirubin, when oxidized, reverts to become biliverdin once again. This cycle, in addition to the demonstration of the potent antioxidant activity of bilirubin, has led to the hypothesis that bilirubin's main physiologic role is as a cellular antioxidant.

Erythrocytes generated in the bone marrow are disposed of in the spleen when they get old or damaged. This releases hemoglobin, which is broken down to heme as the globin parts are turned into amino acids. The heme is then turned into unconjugated bilirubin in the macrophages of the spleen. This unconjugated bilirubin is not soluble in water. It is then bound to albumin and sent to the liver.

In the liver it is conjugated to glucuronic acid, making it soluble in water. Much of it goes into the bile and thus out into the small intestine. Some of the conjugated bilirubin remains in the large intestine and is metabolised by colonic bacteria to urobilinogen, which is further metabolized to stercobilinogen, and finally oxidised to stercobilin. This stercobilin gives feces its brown color. Some of the urobilinogen is reabsorbed and excreted in the urine along with an oxidized form, urobilin.

Normally, a tiny amount of bilirubin is excreted in the urine, accounting for the light yellow color. If the liver's function is impaired or when biliary drainage is blocked, some of the conjugated bilirubin leaks out of the hepatocytes and appears in the urine, turning it dark amber. The presence of this conjugated bilirubin in the urine can be clinically analyzed, and is reported as an increase in urine bilirubin. However, in disorders involving hemolytic anemia, an increased number of red blood cells are broken down, causing an increase in the amount of unconjugated bilirubin in the blood. As stated above, the unconjugated bilirubin is not water soluble, and thus one will not see an increase in bilirubin in the urine. Because there is no problem with the liver or bile systems, this excess unconjugated bilirubin will go through all of the normal processing mechanisms that occur (e.g., conjugation, excretion in bile, metabolism to urobilinogen, reabsorption) and will show up as an increase in urine urobilinogen. This difference between increased urine bilirubin and increased urine urobilinogen helps to distinguish between various disorders in those systems.

Unconjugated hyperbilirubinaemia in a neonate can lead to accumulation of bilirubin in certain brain regions, a phenomenon known as kernicterus, with consequent irreversible damage to these areas manifesting as various neurological deficits, seizures, abnormal reflexes and eye movements. The neurotoxicity of neonatal hyperbilirubinemia manifests because the blood-brain barrier has yet to develop fully, and bilirubin can freely pass into the brain interstitium, whereas more developed individuals with increased bilirubin in the blood are protected. Aside from specific chronic medical conditions that may lead to hyperbilirubinaemia, neonates in general are at increased risk since they lack the intestinal bacteria that facilitate the breakdown and excretion of conjugated bilirubin in the feces (this is largely why the feces of a neonate are paler than those of an adult). Instead the conjugated bilirubin is converted back into the unconjugated form by the enzyme β-glucuronidase and a large proportion is reabsorbed through the enterohepatic circulation.

C. Derivatives

The difference in IC₅₀ of BV and BR for the NS3/4a protease is at least 10-fold (see Examples, Table 2). The compounds differ only in the presence of a double C—C bond at the central methene bridge in BV as compared to single bond in BR.

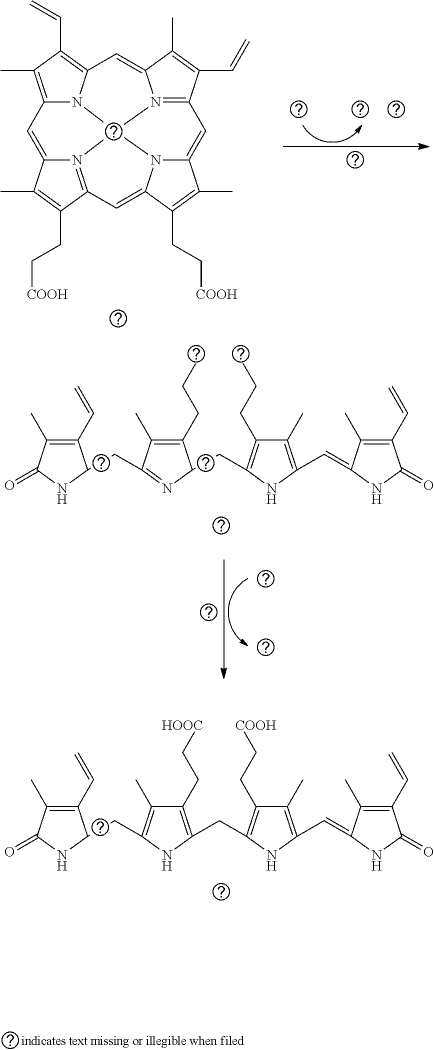

Consequently, BV is fixed centrally, allowing no free rotation about this bond, and its increased IC₅₀ would indicate strongly that this double bond is important for active site binding and antiprotease activity. Functional derivatives of the flanking carboxylic acid moieties are therefore contemplated, particular for the design of irreversible or covalent inhibitors.

D. Protein Purification

It may be desirable to purify BV, BR or derivatives thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, hydrophobic interaction chromatography, exclusion chromatography; polyacrylamide gel electrophoresis; isoelectric focusing. A particularly efficient method of purifying peptides is fast protein liquid chromatography (FPLC).

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, about 96%, about 97%, about 98%, about 99% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme.

E. Sources

Biliverdin is available from Frontier Scientific as either biliverdin hydrochloride (Cat. No. B655-9) or biliverdin dimethyl ester (Cat No. B610-9). Biliverdin hydrochloride is also available from Eschelon Biosciences Inc. (Cat. No. F-H100) and MP Biomedicals (Cat. No. 194886).

Bilirubin is available from Eschelon Biosciences Inc. (Cat. No. F-H120), and from Frontier Scientific as bilirubin (alpha) (Cat No. B584-9), bilirubin dimethyl ester (B612-9), bilirubin conjugate (Cat. No. B850) and mesobilirubin (M589-9).

The compounds are also easily isolated from lipophilic extracts of animal liver. Furthermore, the natural precursor of BV, heme, is available commercially in large quantities as the drug Panhematin (Ovation Pharmaceuticals, Deerfield, Ill.). BV can be easily prepared in large quantities from reaction mixtures of heme and heme oxygenase (Sigma) and HPLC of solvent extracts of the reaction products.

### III. TREATMENT OF HCV INFECTION

A. Formulations

The invention provides for pharmaceutical compositions comprising BV, BR and derivatives thereof. Pharmaceutical compositions are defined as comprising one or more such compounds and a physiologically acceptable carrier, diluent, buffer, carrier or excipient. While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous, intradermal or intramuscular administration. For parenteral administration, such as subcutaneous injection, the carrier preferably comprises water, saline, alcohol, a fat, a wax or a buffer.

In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption or penetration across the blood-brain barrier of the delivered molecule. Such excipients are those substances usually and customarily employed to formulate dosages for parenteral administration in either unit dose or multi-dose form or for direct infusion into the CSF by continuous or periodic infusion from an implanted pump.

Such compositions may also comprise buffers (e.g., neutral buffered saline or phosphate buffered saline), carbohydrates (e.g., glucose, mannose, sucrose or dextrans), mannitol, proteins, peptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site, such as a site of surgical excision of a tumor. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

B. Administration

BV and BR have been used in numerous animal models as protective agents against, sepsis, vascular injuries, and solid organ transplantation, (Ollinger et al., 2007). Because of relatively poor oral bioavailability, initial studies with BV and BR explore intravenous administration. Test dosages may be approximated from animal studies and these compounds are well-tolerated. Biliverdin was administered to rats at levels of 50 mg/kg and prevented vascular injury (Nakao et al., 2005). At levels of 35 mg/kg, BV protected rats against LPS induced septic shock (Sarady-Andrews et al., 2005). At these dosages, BR concentrations are achieved at low pharmacological levels. Thus, 20-200 mg/kg and 25-100 mg/kg are particular dosages contemplated.

The compositions of the present invention may be administered in any suitable manner, often with pharmaceutically acceptable carriers as discussed above, although more than one route can be used to administer a particular composition, and particular route can provide a more immediate and more effective response than another route.

The dose administered to a patient, in the context of the present invention, should be sufficient to effect a beneficial therapeutic response in the patient over any period of time, or to inhibit disease progression. Thus, the composition is administered to a subject in an amount sufficient to alleviate, reduce, cure or at least partially arrest symptoms and/or complications from the disease. An amount adequate to accomplish any of these is defined as a “therapeutically effective dose.” Specifically contemplated are reduced symptoms of viral expression, reduced viral replication and/or reduced viral load.

Routes and frequency of administration of the therapeutic compositions disclosed herein, as well as dosage, will vary from individual to individual, and may be readily established using standard techniques. In general, the pharmaceutical compositions may be administered, by injection (e.g., intracutaneous, intramuscular, intravenous or subcutaneous), intranasally (e.g., by aspiration) or orally. More specifically, between 1 and 100 doses may be administered over a 52 week period. Particularly, 6, 8, 10, 12, 14, or 16 doses are administered, at intervals of 1 week, and additional administrations may be given periodically thereafter. Alternate protocols may be appropriate for individual patients. In one embodiment, two intravenous injections of the composition are administered 7 days apart.

C. Combination Therapy

The use of combination treatments is a common therapeutic approach. This can have the benefit of enhancing therapeutic efficacy of the combined agents, and/or reducing the amount of drug needed to achieve the same benefit as compared to either drug alone, while simultaneously reducing side effects therefrom. Such combinations may involve another anti-HCV treatment that precedes, is co-current with and/or follows the therapies described herein, by intervals ranging from minutes to weeks. In embodiments where the treatment according to the present invention and other therapy are applied separately to a cell, tissue or organism, one would generally ensure that a significant period of time did not expire between the time of each delivery, such that the first and second treatments would still be able to exert an advantageously combined effect on the cell, tissue or organism. For example, in such instances, it is contemplated that one may contact the cell, tissue or organism with multiple modalities substantially simultaneously (i.e., within less than about a minute). In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering a treatment according to the present invention.

Various combination regimens of BV, BR or derivatives and one or more agents may be employed. Non-limiting examples of such combinations are shown below, wherein the BV, BR or derivatives treatment is “A” and the other agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

What follows is a discussion of various HCV therapies than can be used with the treatments according to the present invention.

As discussed above, interferon α-2b (IFN) when used as monotherapy results in sustained response rates of only 10% to 25% of patients with HCV. Retreatment of primary non-responders with IFN alone is unsuccessful in most cases. Retreatment for relapsed patients with interferon α-2b in combination with ribavirin are very promising, but efficacy of combination therapy in primary non-responders is discussed controversially.

Alternative approaches for non-responders might be high dose interferon induction therapy (10 MU QD) in combination with ribavirin or triple therapy with IFN, ribavirin and amantadine. PEGASYS™ (Roche) is a pegylated interferon which can be used as a monotherapy or with COPEGUS™, i.e., ribavirin. Milk thistle is an alternative medicine treatment that has some support in the mainstream medical community.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Materials. Taq DNA polymerase (Perkin-Elmer Cetus, Norwalk, Conn.), and Moloney murine leukemia virus reverse transcriptase (Gibco/BRL Life Technologies, Gaithersburg, Md.) were used in these studies. Bile pigments were purchased from Frontier Scientific, Inc (Logan, Utah) and included bilirubin-IX-α (#B584-9), biliverdin-IX-α hydrochloride (#B655-9) and mesobilirubin (B588-9). Bilirubin mixed isomers, (>99%) was purchased from Sigma Chemical Co (Saint Louis, Mo.). All preparations of tetrapyrroles were the purest form available (99% purity). The BR mixed isomer preparation contained 93% bilirubin IX-α, 3% bilirubin III-α, 3% bilirubin XIII-α and traces of β and γ isomers (MSDS information). BV was prepared by oxidation of highly purified α-bilirubin followed by final chromatographic purification (personal communication, Dr. Colin Ferguson, Echo Laboratories, Frontier Scientific, Salt Lake City, Utah). All tetrapyrroles were dissolved in 0.2 N NaOH and added in small volumes to achieve the final concentration. Controls received an identical volume of diluted NaOH only. HCV protease assay kits (SensoLyte 620, Cat#71146) and recombinant NS3/4A protease (Ac-DEDif-EchaC, Cat #25346) were purchased from AnaSpec.

Antibodies. Antibody to human biliverdin reductase (BVR) and all secondary antibodies were purchased from Santa Cruz Biotechnology (Santa Cruz, Calif.) unless indicated otherwise.

Cell lines and cell culture. The human hepatoma cell line (Huh5-15) with replicating sub-genomic HCV RNA (Lohmann et al., 1999) was a kind gift of Dr. Volker Lohmann (Institute for Virology, Johannes-Gutenberg University, Mainz, Germany), and cultivated as described (Zhu et al., 2008). Huh7.5 cells harboring full length (Huh7.5FL) Conl replicons (Blight et al., 2002) were a kind gift of Dr. Charles Rice (Rockefeller University, New York, N.Y.). These cells were passed as recommended by their laboratory of origin (Blight et al., 2002). An infectious clone of HCV, J6/JFH, was inoculated into Huh7.5 cells and the cultures passed as previously described (Lindenbach et al., 2005). Cells were incubated with BV, BR, or FeCl₂ for 24-48 h in DMEM containing 5% FBS.

Quantitative Real-time RT-PCR. Total RNA was extracted from cells using Trizol reagent (Invitrogen, Carlsbad, Calif.), treated with Turbo RNase free DNase (Ambion, TX), and processed as described (Zhu et al., 2008). To assess replication, the primers and probe designed by Takeuchi et al. (1999) targeting sequences located in 5′UTR (nt130-290) were employed as described for each replicon line (Blight et al., 2002; Lohmann et al., 2003). Real-time RT-PCR was performed using Taq DNA polymerase with the TaqMan Universal PCR Master Mix Protocol (Perkin Elmer Applied Biosystems, Foster City, Calif.). Quantitation was performed using the comparative cycle threshold (ΔC_{T}) method as described previously (Abdalla et al., 2004).

Immunocytochemical staining. Cells were fixed in absolute methanol, washed in PBS, and incubated with positive HCV genotype 2A polyvalent human serum. On western blots, this antiserum specifically recognized core, NS3, and NS5A at their appropriate mobility's. Antibody binding was evaluated following labeling with anti-human secondary antibody-alkaline phosphatase conjugate and results recorded by photomicroscopy.

Western blot analysis. Western blots (WB) were performed as previously described using enhanced chemiluminescence for signal detection (ECLTM, Amersham) (Abdalla et al., 2005). Signal intensities were quantified using Image J software (NIH).

Biliverdin reductase (BVR) knockdown. Biliverdin reductase (BVR) siRNA and control (scrambled) siRNA were purchased from Santa Cruz Biotechnology (sc-44650 and sc-37007). BVR knockdown was performed as described previously (Ryter et al., 2007). Efficiency of the knockdown was monitored by semiquantitative densitometry of BVR WB.

In vitro assay of HCV NS3/4A recombinant protease. Protease activity was determined fluorometrically with the SensoLyte 620 HCV Protease Assay (AnaSpec, Fremont, Calif.) using a wide wavelength excitation/emission (591 nm/622 nm respectively) fluorescence energy transfer peptide according to the manufacturer's instructions. Control incubations with BV or metabolite only were performed to eliminate or correct for autofluorescence or quenching. A competitive inhibitor of the NS3/4A protease, AnaSpec #25346, was used as positive control.

For assays employing endogenous NS3/4A protease, the high-throughput FRET assay was used with modifications (Yu et al., 20009). Replicon cells at 90% confluence were lysed in ice-cold 1.25% Triton X-100 lysis buffer (Yu et al., 20009). Serial dilutions of lysates were added in triplicate to black 96-well plates, together with designated concentrations of tetrapyrrole. The HiLyte Flur™ TR/QXL™ 610 substrate solution (AnaSpec) was prepared according to the manufacturer's instructions. The plate and substrate were incubated at 37° C. for 15 min, and then 50 μl substrate solution was added to each well. The plates were incubated at room temperature for 30 min, avoiding direct light. The fluorescence intensity was then measured as above for recombinant enzyme assays using FLUOstar Optima (BMG Lab Tech, Cary, N.C.). To ensure specificity for the NS3/4A protease, lysates of parental Huh7 and Huh7.5 cells were run in every assay and the results subtracted as background fluorescence.

Immunoprecipitation of NS5A. Log-phase replicons were treated with various concentrations of BV for 48 hr, washed, lysed in cell lysis buffer (Cell Signaling Technology, Beverly, Mass.) and clarified by cold centrifugation (14,000×g for 10 min). An aliquot of supernatant containing 100 μg protein was incubated with 1 μg of HCV NS5A monoclonal antibody (Meridian Life Science, Saco, Me.) and 20 μl of rProtein G Agarose (Invitrogen, CA) overnight at 4° C. The agarose was collected by 3000×g spin, washed three times with ice-cold PBS, then dissolved in 40 μL 2× Laemmli sample buffer (Bio-Rad, CA) and assayed by WB.

Cytotoxicity assay. Cytotoxicity was assessed using the MTT assay as detailed previously (Wen et al., 2008). Cells (1×10⁴ per well) were seeded in 96-well culture plates and allowed to attach overnight. Treatments with BV and BR were done in medium containing 5% FBS and employed the same concentrations used to assess antiviral activity.

Statistical analysis. Data from individual experiments as well as combined data from separate experiments were expressed as mean+/−standard error of the mean. The significance between means was determined using Student's t-test and when applicable, with ANOVA using pooled variances. P values less than 0.05 were considered significant. All experiments were repeated at least three times.

### Example 2

### Results

The inventors previously showed that induction of HO-1 with hemin results in decreased HCV replication in vitro (Zhu et al., 2008); however, it was not known whether physiological concentrations of heme exert antiviral effects. Incubation of replicons with various amounts of hemin demonstrated a concentration-dependent antiviral effect of hemin, apparent at levels as low as 5 (Table 1). These concentrations are well within the physiological range of heme in human circulation (10-16 μM) and, in the presence of HO-1, would be expected to yield equimolar quantities of BV, Fe and carbon monoxide.

TABLE 1
Heme inhibition of HCV replication

| | Heme | Relative | |
|---|---|---|---|
| Replicon | [uM] | [HCV] [ΔC_{T}] | *SEM |
| Huh5-15NS | 0 | 1.0 | 0.08 |
| | 5 | 0.27 | 0.02 |
| | 10 | 0.09 | 0.003 |
| | 20 | 0.03 | 0.003 |
| Huh7.5FL | 0 | 1.0 | 0.16 |
| | 5 | 0.22 | 0.03 |
| | 10 | 0.08 | 0.02 |
| | 20 | 0.04 | 0.006 |

Log phase replicon cells were incubated with hemin or control vehicle overnight and the relative amount of HCV RNA then determined by the comparative cycle threshold level (ΔCT) as described in Methods. Each value is the mean of four determinations.
*p < 0.01 all within group difference.

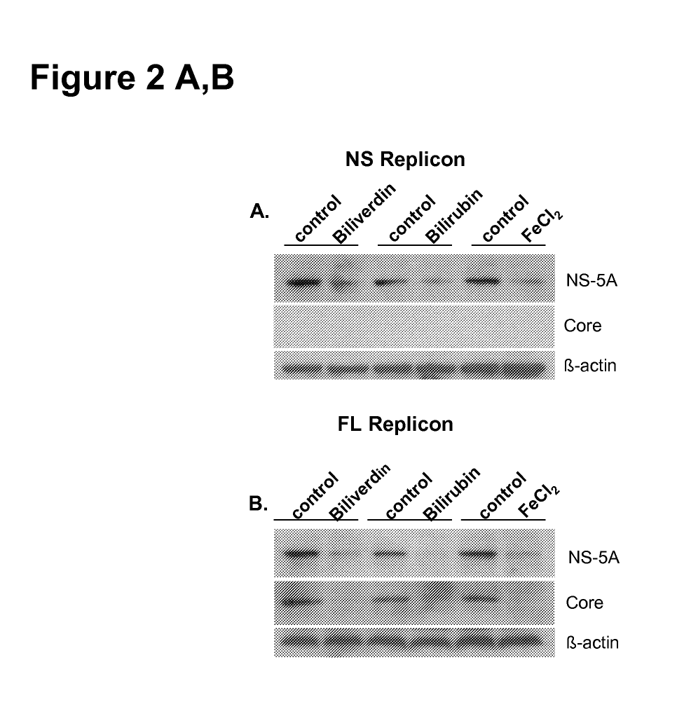

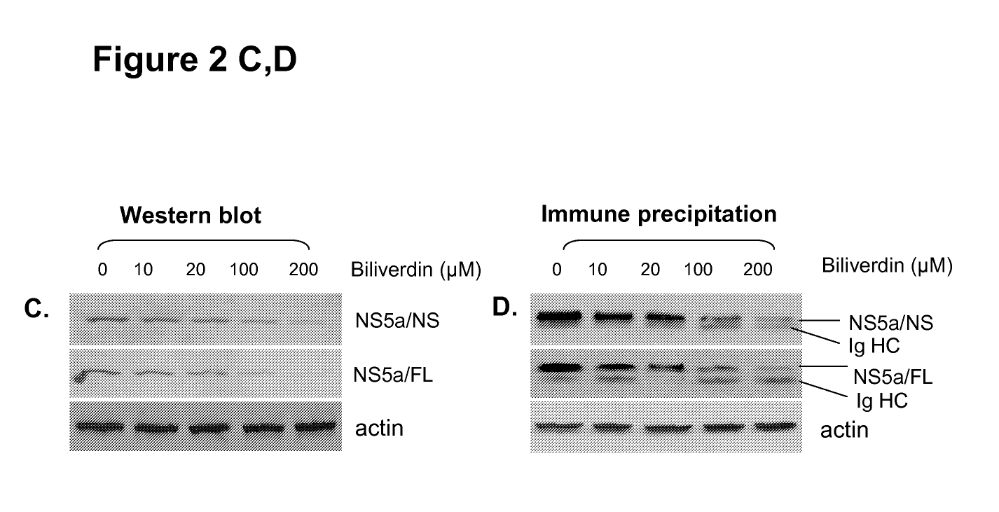

Antiviral activity of BV and BR. The inventors next tested BV and isomers of its metabolite BR for antiviral activity in HCV full-length and nonstructural replicons. In both replicon lines, BV showed significant antiviral activity at concentrations as low as 20 μM. In contrast, concentrations of BR-IX-α or BR mixed isomers required to suppress HCV replication were considerably higher (200 μM) (FIGS. 1A-D). For comparison, 20 μM of BV or BR corresponds to a circulating BR level of about 1.4 mg/dl. Western blots (FIGS. 2A-B) confirmed decreased NS5A in both replicon lines following treatment with BV or BR. Levels of core protein were also reduced by BV or BR in full-length replicons, consistent with reduced replication of HCV. Treatment with BV dose-dependently decreased NS5A when assayed by WB (FIG. 2C) or immunoprecipitation using specific NS5A antibody (FIG. 2D), In accord with prior reports (Fillebeen et al., 2005; Yuasa et al., 2006), FeCl₂ (100 mM) also decreased NS5A and core protein (FIGS. 2A-B) as well as diminishing HCV RNA (not shown).

The inventors next evaluated the cytotoxicity of BV in the Huh7.5 replicons. Treatment of replicon or parental cell lines with 20-200 μM BV in serum-free culture medium for 24-48 hr was associated with modest toxicity (15%, as determined with MTT assay); however, toxicity was eliminated by the inclusion of 3-5% FBS (data not shown). Consequently, all cellular assays with BV or BR were done in the presence of 5% FBS.

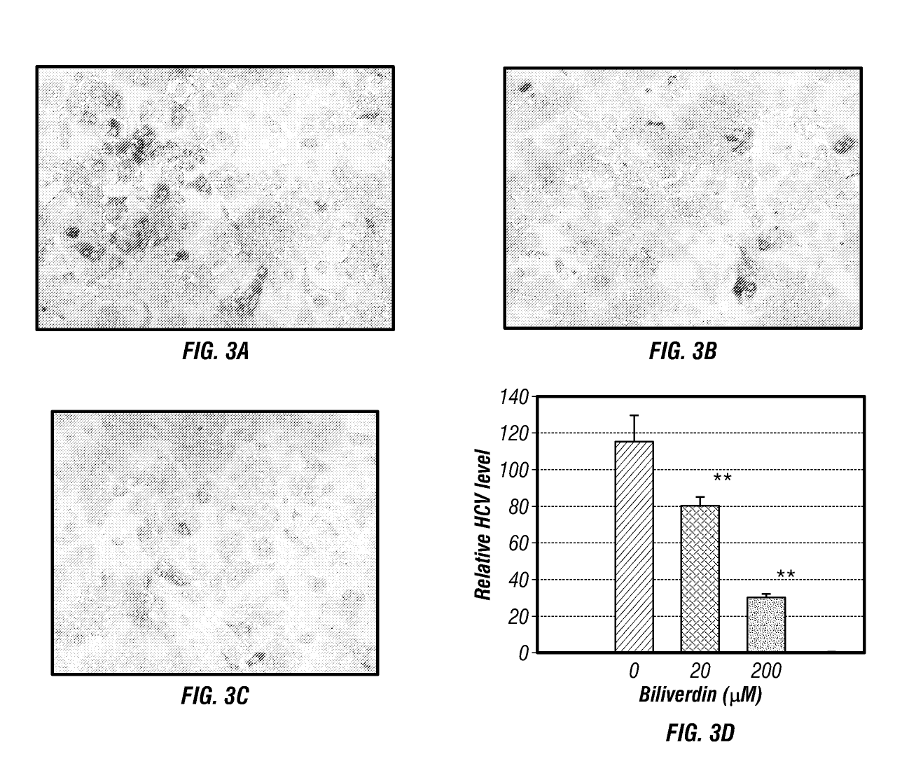

They next tested the effects of BV (20-200 μM) on HCV infection of Huh7.5 cells with J6/JFH infectious HCV construct (Lindenbach et al., 2005). BV markedly decreased Huh7.5 cell infection with J6/JFH, based on immunoreactivity of HCV polyvalent sera (FIGS. 3A-C) and measurement of HCV RNA (FIG. 3D).

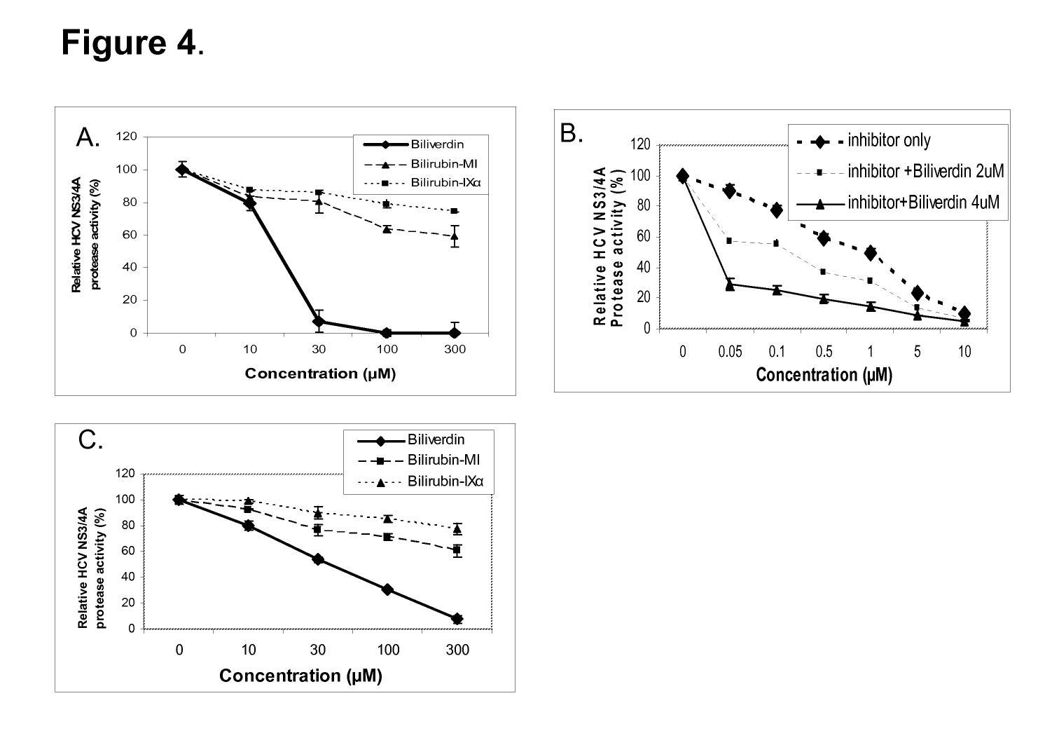

Biliverdin inhibits NS3/4A protease. Deconjugated bile pigments are known to inhibit serine-activated pancreatic proteases such as chymotrypsin and trypsin (Qin, 2007). This led the inventors to evaluate the effects of BV and other tetrapyrroles on the HCV NS3/4A protease (FIGS. 4A-C). These assays were conducted with wide wavelength excitation/emission (591 nm/622 nm, respectively) transfer peptides. Preliminary experiments established that shorter fluorescence wavelength transfer peptides (340 nm/490 nm or 490 nm/520 nm, excitation/emission, respectively) could not be employed because BV, BR, and other tetrapyrroles showed unacceptable autofluorescence and/or quenching at the shorter wavelengths.

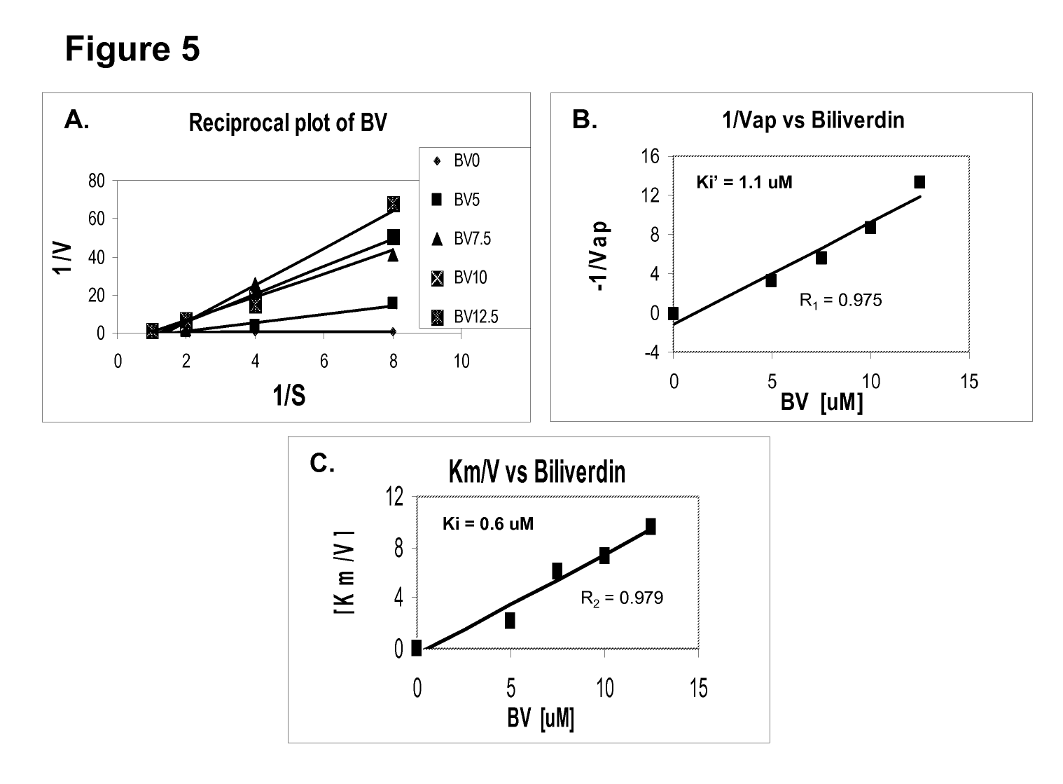

In an assay utilizing a recombinant protease, BV was a markedly more potent inhibitor than BR (either highly purified BR-IXα or BR mixed isomers) (FIG. 4A). BV also displayed the highest IC₅₀ (9.3 μM) of any tetrapyrrole tested (Table 2), which was similar to that of the commercial NS3/4A inhibitor, AnaSpec #25346. Notably, the IC₅₀ value for the commercial inhibitor in the inventors' hands (4.9 μM) is indistinguishable from the value reported by the manufacturer (5 μM), supporting the accuracy of the assay. Assays conducted in the presence of both BV and #25346 showed an additive effect (FIG. 4B), indicating a mixed inhibitory mechanism of BV on the NS3/4A protease as described below (FIGS. 5A-C). A modification of the fluorescence protease assay was also performed in which freshly prepared protease from replicons was used in place of recombinant protease, as described by Yu et al. (2009) (FIG. 4D). The results of these experiments were similar to those with the recombinant enzyme, although inhibition of the endogenous protease required slightly higher concentrations of BV than the recombinant enzyme, possibly due to conversion of BV to BR by endogenous BVR in the microsomes.

TABLE 2
Activity of biliverdin and derivatives for
Inhibition of HCV NS3/4A protease as compared
to known competitive inhibitor AnaSpec #25346

| Test Compound | IC₅₀ (μM) |
|---|---|
| AnaSpec #25346 | 4.9 |
| Biliverdin | 9.3 |
| Bilirubin (mixed | >300 |
| isomers) | |
| Bilirubin IXα | >300 |
| Mesobilirubin | >300 |
| Biliverdin dimethylester | >300 |

Protease activity was determined fluorometrically with the SensoLyte 620 HCV Protease Assay using a wide wavelength excitation/emission (591 nm/622 nm) fluorescence energy transfer peptide. Concentrations of inhibitor required for 50% inhibition (IC₅₀) were determined by regression analysis.

The kinetics of BV inhibition of NS3/4A protease was assessed on Lineweaver-Burk Plots (FIG. 5A). These data indicated that BV competitively inhibits NS3/4A protease, based on the characteristic increase in slope with higher concentrations of inhibitor. Slopes (Km/V) and y intercepts (1/Vmax) of the primary reciprocal plots were then used to make secondary plots (FIGS. 5B-C) to estimate Ki and Ki′, respectively, as general indices of competitive and noncompetitive inhibition. Note that plots of BV vs either 1/Vap or Km/V showed highly significant linearity, (r₁=0.975 and r₂=0.979 respectively, P<0.005) suggesting that BV has both noncompetitive and competitive inhibitor activity for NS3/4A protease (Ki′=1.1 and Ki=0.6 uM, respectively).

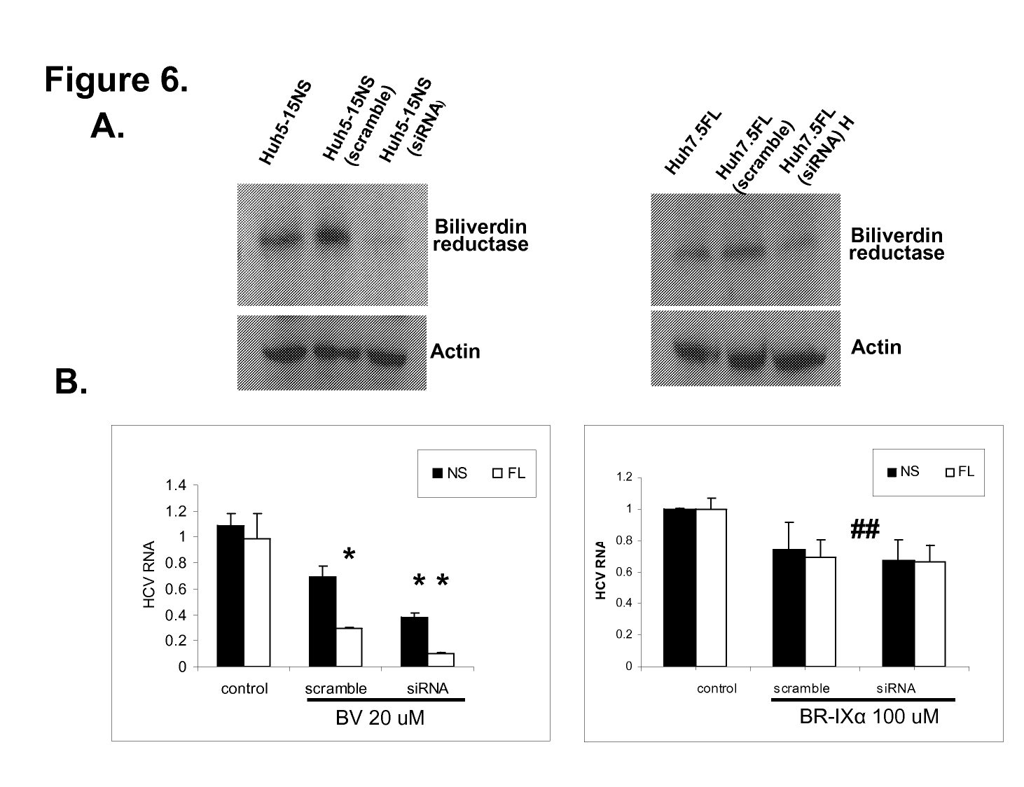

BVR knockdown. BV is rapidly reduced to BR by the soluble enzyme BVR (Ryter et al., 2006). The inventors hypothesized that knockdown of BVR expression would result in increased antiviral activity for BV by diminishing its conversion to the less potent BR. Preliminary WB showed that knock down of BVR was highly efficient and led to >80% reduction of BVR expression in both replicon lines (FIG. 6A). The antiviral activity of BV was significantly enhanced by BVR knockdown compared to control (scramble) RNA knockdown (FIG. 6B, left panel, p<0.01). In contrast, knockdown of BVR prior to incubation of replicons with BR had no significant effect on the relatively modest antiviral activity of BR (FIG. 6B, right panel). Taken together, these data support the concept that BVR knockdown augments the antiviral activity of BV by arresting its conversion to BR and thereby maintaining higher intracellular levels of BV.

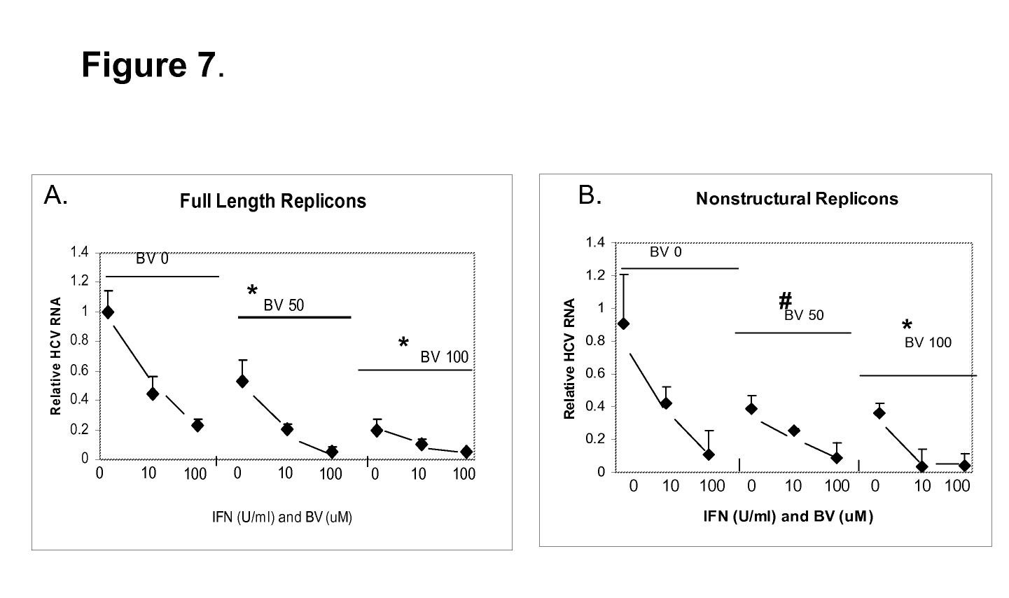

Because interferon remains a cornerstone of HCV therapy, The inventors examined the effects of BV on the antiviral activity of α-interferon. As shown in FIGS. 7A-B, BV had a clear additive effect when exposed to cells in the presence of interferon. These findings indicate that BV does not appear to compromise the action of interferon, but rather to enhance it. They also raise the possibility that the BV or stable derivatives could be used as antiprotease agents in combination with interferon.

### Example 3

### Discussion

Heme oxygenase catalyzes the breakdown of heme to equimolar quantities of BV, iron and carbon monoxide. Expression of the inducible isoenzyme HO-1, also known as heat shock protein 32, is readily upregulated in response to stressors such as hypoxia, heat shock, heavy metals, and oxidants (Ryter et al., 2006). Along with other investigators, the inventors have shown that HO-1 expression is downregulated in HCV-infected human liver and highly modulated in some in vitro models of HCV (Abdalla et al., 2004; Zhu et al., 2008; Abdalla et al., 2005; Wen et al., 2008; Ghaziani et al., 2006; Shan et al., 2007). Furthermore, in cell culture models of HCV, HO-1 modulates both oxidative stress and HCV replication (Zhu et al., 2008; Shan et al., 2007).

In order to identify the mechanisms by which exogenous heme or HO-1 overexpression inhibits HCV replication in replicons (Zhu et al., 2008; Shan et al., 2007; Fillebeen et al., 2007), the inventors studied the antiviral activities of heme oxidation products. Two reports have addressed the ability of iron to inhibit HCV replication (Fillebeen et al., 2005; Yuasa et al., 2006), however, little attention has been directed at the other heme degradation products, BV and carbon monoxide. These data demonstrate that BV has potent antiviral activity against HCV in two separate replicon lines and also inhibits replication in J6/JFH construct-infected Huh7.5 cells. Most importantly, these findings provide evidence that BV is a potent inhibitor of the HCV NS3/4A protease. In addition to the inventors' preliminary data (Zhu et al., 2008; Zhu et al., 2009), Lehman et al. (2010) recently reported that BV has antiviral activity in replicon cells and noted that antiviral activity was accompanied by a rise in specific interferon stimulated gene (ISG) products. These observations are consistent with the inventors' data showing that BV inhibits NS3/4A protease. Thus, the inventors propose that the rise in ISGs is a direct result of NS3/4A inactivation by BV, which prevents cleavage of the adapter molecules TRIF and IPS-1, thereby restoring TLR3 and RIG-I signaling for innate interferon production (Foy et al., 2003; Foy et al., 2005). Work is currently underway to explore this possibility further.

Iron has also been shown to inhibit HCV replication through prevention of divalent cation binding to RdRp (Fillebeen et al., 2005; Yuasa et al., 2006). The results reported here showing that FeCl₂ inhibits replication (FIGS. 2A-D) support these data. Thus, the identification of BV as a strong antiviral agent with activity against the NS3/4A protease demonstrates that heme oxidation by HO-1 liberates at least two antiviral agents, iron and BV. These potent antiviral effects may explain the downregulation of HO-1 by HCV in infected human liver, in contrast to other liver diseases where HO-1 is frequently upregulated (Abdalla et al., 2004). Importantly, the antiviral activity of heme is apparent at physiological serum concentrations, raising the possibility that heme and/or BV could be used as specifically targeted antiviral compounds (STAT-C). Heme (as hemin) is already commercially available for treatment of the porphyrias. Although antiviral activities of BV have not been formally addressed in vivo, the compound appears safe and has been shown to prevent hepatic reperfusion injury and vascular injury-induced intimal hyperplasia in rodent models (Nakao et al., 2005).

Since the discovery of HCV, the NS3/4A protease has been an attractive target for antiviral therapies. Structurally, the enzyme is a typical β-barrel serine-activated protease with a canonical Asp-His-Ser catalytic triad (Love et al., 1996; Yan et al., 1998). Both boceprevir and telaprevir, two promising antiviral agents currently in phase III trials, utilize an α-ketoamide functional group as a “serine trap” to bind and slowly dissociate from the catalytic serine in the enzyme's active site. However, BV does not contain an α-ketoamide moiety and work is currently underway to determine the chemical structure(s) important for its interaction with the protease. Inhibition appears complex since kinetic studies showed a mixed competitive and non-competitive mechanism. Consequently, in addition to competitive binding to the substrate active site, BV may exert allosteric effects on enzyme activity, possibly through the known antioxidant or solvent effects of tetrapyrroles (McDonagh, 2001).

The HO reaction releases nearly exclusively BV-IX-α (Noguchi et al., 1979), which is then reduced to BR-IX-α (Tenhunen et al., 1970), the predominant BR isomer produced by adult mammalian liver. The fact that highly purified BR-IX-α and mixed isomers of BR, are much weaker inhibitors of NS3/4A protease than BV suggests that BR is unlikely to exert antiviral activity in vivo at normal BR blood levels. Interestingly, BV differs from BR by a lone carbon-carbon double-bond at position 10. It is intriguing that this single difference causes such a profound difference in the IC₅₀ values of the two compounds (9 μM vs >300 μM, respectively) (Table 2). The inventors speculate that the fixed planar double-bond at position 10 may be crucial for active site binding and they are pursuing this further with structure-function studies of BV.

The inhibition of NS3/4A protease by BV, and to a lesser extent BR and other tetrapyrroles is not without precedence. In the bowel, unconjugated BR, but not BV, inhibits chymotrypsin and trypsin in a dose-related fashion at similar concentrations to those reported here for antiviral activity (Qin, 2007). In contrast, BV and BR inhibit HIV protease with nearly equivalent potency (McPhee et al., 1996), while BV has been shown to decrease viral activity of herpesvirus 6 in vitro (Nakagami et al., 1992).

In summary, the inventors have evaluated the antiviral activity of BV, the primary tetrapyrrole product of heme oxidation. Their findings demonstrate that BV is a potent antiviral agent, likely as a consequence of its ability to inhibit the NS3/4A protease. These findings suggest that heme, BV, or related derivatives may be useful for future drug therapies targeting the NS3/4A protease.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### V. REFERENCES

The following references, to the extent that they provide exemplary procedural or other details supplementary to those set forth herein, are specifically incorporated herein by reference.
Abdalla et al., J. Infect. Dis., 190(6):1109-1118, 2004.
Abdalla et al., J. Med. Virology, 76(4):489-497, 2005.
Alter et al., N Engl. J. Med., 341:556-562, 1999.
Blight and Rice, J. Virol., 71:7345-7352, 1997.
Blight et al., J. Virology, 76(24):13001-13014, 2002.
Choo et al., Proc. Natl. Acad. Sci. USA, 91:1294-1298, 1994.
Choo et al., Science, 244:359-362, 1989.
Di Bisceglie, Lancet., 351:351-355, 1998.
Eigen, Scientific American, 269:42-49, 1993.
Fan and Di Bisceglie, J. Med. Virol., 70:212-218, 2003.
Fenner et al., In: The Biology of Animal Viruses, 2^{nd} Ed., 417-418, Academic Press, London, 1974.
Ferrari et al., J. Virology, 73(2):1649-1654, 1999.
Fillebeen et al., J. Biol. Chem., 280(10):9049-9057, 2005.
Fillebeen et al., J. Hepatology, 47(1):12-22, 2007.
Foy et al., Proc. Natl. Acad. Sci. USA, 102(8):2986-2991, 2005.
Foy et al., Science, 300(5622):1145-1148, 2003.
Ghaziani et al., J. Hepatology, 45(1):5-12, 2006.
Handbook of Pharmaceutical Salts: Properties, and Use (Stahl & Wermuth, Eds.), Verlag Helvetica Chimica Acta, 2002.
Hijikata et al., Biochem. Biophys. Res. Commun., 175:220-228, 1991.
Holland et al., Science, 215:1577-1585, 1982.
Immenschuh and Ramadori, Biochem. Pharmacol., 60:1121-1128, 2000.
Ishii et al., Hepatology, 28:1117-1120, 1998.
Kato et al., Biochem. Biophys. Res. Commun., 189:119-127, 1992.
Klenerman and Zinkernagel, Nature, 394:482-485, 1998.
Kolykhalov et al., J. Virol., 70:3363-3371, 1996.
Krawczynski et al., J. Infect. Dis., 173:822-828, 1996.
Kuntzen et al., Hepatology, 48:1769-1778, 2008.
Lagging et al., J. Virol., 72:3539-3546, 1998.
Lehmann et al., Hepatology, 51(2):398-404, 2010.
Lemon et al., Hepatology, 31:800-806, 2000.
Lindenbach et al., Science, 309(5734):623-626, 2005.
Lohmann et al., J. Virology, 77(5):3007-3019, 2003.
Lohmann et al., Science, 285(5424):110-113, 1999.
Love et al., Cell, 87(2):331-342, 1996.
McDonagh, Nature Struct. Biol., 8(3):198-200, 2001.
McPhee et al., Biochem. J., 320:681-686, 1996.
Mehta et al., Lancet., 359:1478-1483, 2002.
Moorman et al., Archivum Immunologiae et Therapiae Experimentalis, 49:189-194, 2001.
Nakagami et al., Microbiol. Immunol., 36(4):381-390, 1992.
Nakao et al., Circulation, 112:587-591, 2005.
Natl. Institutes of Health Consensus Dev. Conf. Panel, 1997.
Neumann et al., Science, 282:103-107, 1998.
Noguchi et al., Febs Lett., 98(2):281-284, 1979.
Ollinger et al., Antioxidants & Redox Signaling, 9:2175-2185, 2007.
Qin, Gut, 56(11):1641-1642, 2007.
Robertson et al., Arch. Virol., 143:2493-2501, 1998.
Rosenberg, Gut., 44:759-764, 1999.
Ryter and Tyrrell, Free Rad. Biol. Med., 28:289-309, 2000.
Ryter et al., Am. J. Resp. Cell Molec. Biol., 36(2):175-182, 2007.
Ryter et al., Physiological Rev., 86(2):583-650, 2006.
Sarady-Andrews et al., Amer. J. Physiology-Lung Cell. Molec. Physiol., 289:L1131-L1137, 2005.
Shan et al., Gastroenterology, 132(4):A824, 2007.
Shan et al., Gastroenterology, 133(4):1166-1174, 2007.
Shimizu et al.; J. Virol., 68:1494-1500, 1994.
Simmonds, Hepatology, 21:570-583, 1995.
Takeuchi et al., Gastroenterology, 116(3):636-642, 1999.
Tenhunen et al., Biochemistry, 9(2):298, 1970.
Wen et al., Cell Biol. Toxicol., 24(2):175-188, 2008.
Yamada et al., Virology, 223:255-261, 1996.
Yan et al., Protein Sci., 7(4):837-847, 1998.
Yoshioka et al., J. Infect. Dis., 175:505-510, 1997.
Yu et al., Antimicrob. Agents Chemo., 53(10):4311-4319, 2009.
Yuasa et al., Liver International, 26(9):1111-1118, 2006.
Zhu et al., Hepatology, 48(4):1162A-1163A, 2008.
Zhu et al., Hepatology, 48(5):1430-1439, 2008.
Zhu et al., Hepatology, 50(4):675A, 2009.
Zibert et al., Hepatology, 25:1245-1249, 1997.
Zibert et al., J. Virol., 71:4123-417, 1997.

1. A method for inhibiting hepatitis C virus (HCV) replication comprising contacting an HCV-infected cell with bilirubin, biliverdin or a biliverdin derivative.

2. The method of claim 1, wherein the HCV-infected cell is contacted with bilirubin.

3. The method of claim 1, wherein the HCV-infected cell is contacted with biliverdin.

4. The method of claim 1, wherein the HCV-infected cell is contacted with an HCV-infected cell with biliverdin derivative of the formula: 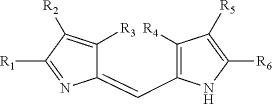
wherein:
R₁ and R₆ are independently alkenyl_{(C≦12)}, aryl_{(C≦12)}, aralkyl_{(C≦12)}, heteroaryl_{(C≦12)}, heteroaralkyl_{(C≦12)}, acyl_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups; and
R₂, R₃, R₄ and R₅ are independently:
hydrogen, hydroxy, halo, amino, nitro, hydroxyamino, cyano, azido or mercapto; or
alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, alkenyloxy_{(C≦12)}, alkynyloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups;
or a pharmaceutically acceptable salt, tautomer, or optical isomers thereof, provided that the biliverdin derivative is not biliverdin.

5. The method of claim 1, further comprising contacting said cell with second agent selected from the group consisting of pegylated interferon, ribavarin or an NS3/4A protease inhibitor.

6. The method of claim 5, wherein said second agent is contacted with said cell at the same time as bilirubin, biliverdin or a biliverdin derivative.

7. The method of claim 5, wherein said second agent is contacted with said cell before or after bilirubin, biliverdin or a biliverdin derivative.

8. (canceled)

9. The method of claim 1, further comprising contacting said cell with bilirubin, biliverdin or a biliverdin derivative at least a second time.

10. The method of claim 1, wherein said cell is contacted with:
(i) bilirubin and biliverdin;
(ii) bilirubin and a biliverdin derivative;
(iii) biliverdin and a biliverdin derivative; or
(iv) bilirubin, biliverdin and a biliverdin derivative.

11. A method for inhibiting hepatitis C virus (HCV) replication in a subject comprising administering to said subject bilirubin, biliverdin or a biliverdin derivative.

12. The method of claim 11, wherein said subject is administered bilirubin.

13. The method of claim 11, wherein said subject is administered biliverdin.

14. The method of claim 11, wherein said subject is administered a biliverdin derivative of the formula: 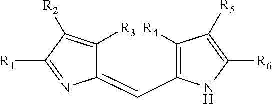
wherein:
R₁ and R₆ are independently alkenyl_{(C≦12)}, aryl_{(C≦12)}, aralkyl_{(C≦12)}, heteroaryl_{(C≦12)}, heteroaralkyl_{(C≦12)}, acyl_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups; and
R₂, R₃, R₄ and R₅ are independently:
hydrogen, hydroxy, halo, amino, nitro, hydroxyamino, cyano, azido or mercapto; or
alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, alkenyloxy_{(C≦12)}, alkynyloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups;
or a pharmaceutically acceptable salt, tautomer, or optical isomers thereof, provided that the biliverdin derivative is not biliverdin.

15. The method of claim 11, further comprising administering to said subject a second agent selected from the group consisting of pegylated interferon, ribavarin or an NS3/4A protease inhibitor.

16. The method of claim 15, wherein said second agent is administered at the same time as bilirubin, biliverdin or a biliverdin derivative.

17. The method of claim 15, wherein said second agent is administered before or after bilirubin, biliverdin or a biliverdin derivative.

18. (canceled)

19. The method of claim 11, further comprising administering to said subject bilirubin, biliverdin or a biliverdin derivative at least a second time.

20. The method of claim 11, wherein said subject is administered:
(i) bilirubin and biliverdin;
(ii) bilirubin and a biliverdin derivative;
(iii) biliverdin and a biliverdin derivative; or
(iv) bilirubin, biliverdin and a derivative of biliverdin.

21. A pharmaceutical formulation comprising:
(a) bilirubin, biliverdin and/or a biliverdin derivative; and
(b) pegylated interferon, ribavarin and/or an NS3/4A protease inhibitor, dispersed in a pharmaceutically acceptable buffer, diluent or excipient.

22. The formulation of claim 21, comprising bilirubin.

23. The formulation of claim 21, comprising biliverdin.

24. The formulation of claim 21, comprising a biliverdin derivative of the formula: 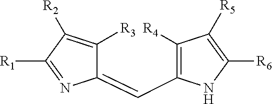
wherein:
R₁ and R₆ are independently alkenyl_{(C≦12)}, aryl_{(C≦12)}, aralkyl_{(C≦12)}, heteroaryl_{(C≦12)}, heteroaralkyl_{(C≦12)}, acyl_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups; and
R₂, R₃, R₄ and R₅ are independently:
hydrogen, hydroxy, halo, amino, nitro, hydroxyamino, cyano, azido or mercapto; or
alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, alkenyloxy_{(C≦12)}, alkynyloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, or a substituted version of any of these groups;
or a pharmaceutically acceptable salt, tautomer, or optical isomers thereof, provided that the biliverdin derivative is not biliverdin.

25. The formulation of claim 21, comprising (a) biliverdin, bilirubin and/or a biliverdin derivative and (b) pegylated interferon and ribavarin.

26-27. (canceled)
